(19) United States
(12) Patent Application Publication
(10) Pub. No.: US 2016/0039868 A1
(43) Pub. Date: Feb. 11, 2016

(54) PROCESS FOR ISOLATION AND STABILISATION OF KEY INTERMEDIATES FOR HIGH EFFICIENCY REFOLDING OF RECOMBINANT PROTEINS

(71) Applicant: BIOGENOMICS LIMITED, Thane (West), Maharashtra (IN)

(72) Inventors: Archana KRISHNAN, THANE (IN); Sanjay SONAR, THANE (IN); NIKHIL GHADE, MUMBAI (IN); FAIZA SHAIKH, (IN)

(73) Assignee: BioGenomics Limited, THANE WEST, MH (IN)

(21) Appl. No.: 14/776,343

(22) PCT Filed: Mar. 21, 2014
(86) PCT No.: PCT/IN2014/000180

§ 371 Date: Sep. 14, 2015

(30) Foreign Application Priority Data
Mar. 22, 2013 (IN) 986/MUM/2013

(51) Int. Cl. C07K 1/113

(52) U.S. Cl. CPC C07K 1/1136

(57) ABSTRACT

A process for obtaining a refolded recombinant protein from an unfolded recombinant protein present in inclusion bodies isolated from wet cells harvested from a cell culture is provided. The process includes a) reducing the inclusion bodies by treating the inclusion bodies with a reducing buffer, to obtain reduced inclusion bodies; b) obtaining stable first intermediate state I₁ of the unfolded recombinant protein present in the reduced inclusion bodies; and c) refolding the stable first intermediate state I₁ of the unfolded recombinant protein, present in the reduced inclusion bodies, in refolding buffer to obtain a second intermediate state I₂ that folds to produce the refolded recombinant protein.

### CROSS REFERENCE TO RELATED APPLICATION

This application claims priority to the pending PCT application PCT/IN2014/000180, filed on 21 Mar. 2014. The pending PCT application PCT/IN2014/000180 is hereby incorporated by reference in its entireties for all of its teachings.

### FIELD OF THE INVENTION

The present invention relates to production of recombinant proteins, and more particularly to refolding of recombinant proteins from inclusion bodies produced in prokaryotic host cells.

### BACKGROUND

Recombinant DNA (rDNA) technology has been used to clone, express and purify several proteins of therapeutic or other economic value such as Insulin, Insulin analogues, trypsin, Granulocyte Colony Stimulating Factor (G-CSF), Granulocyte Macrophage Colony Stimulating Factor (GM-CSF), etc., from prokaryotic as well as eukaryotic cells. However, the use of prokaryotic cells e.g. E. coli is more widespread owing to the better cost-benefit economics of production of recombinant proteins. E. coli bacteria or other prokaryotic host cells are easy to cultivate, since they are capable of producing biomass at a rapid rate. This enables their use in high-cell density fermentations with much better scalability than eukaryotic host cell based fermentations or cell cultures.

The above economic advantages are, however, challenged by the fact that E. coli are unable to perform post-translational modifications of the heterologously expressed proteins. Further, due to increased expression in E. coli, the recombinant proteins have proclivity to aggregate as inclusion bodies. Inclusion bodies (IBs) are the aggregates of insoluble, biologically inactive and unfolded proteins that are produced intracellularly in bacteria. IBs also include the protein of interest or the target protein e.g. Insulin, Insulin analogues, trypsin, Granulocyte Colony Stimulating Factor (G-CSF), Granulocyte Macrophage Colony Stimulating Factor (GM-CSF), etc. It is from IBs that the concentrated target protein is purified. After isolation from the host cell proteins (HCPs), the target protein is refolded or renatured to its biologically active form or conformation. A modest increase in yield of biologically active proteins may lead to substantial commercial benefits. The problems that are usually encountered during the renaturation, isolation and purification of the biologically active recombinant protein include misfolding of proteins, protein loss, protein aggregation etc. This is further complicated by the fact that the traditional processes of obtaining biologically active refolded recombinant proteins are multi-step process that includes treating of inclusion bodies through a number of reagents and subjecting them to a series of processes such as centrifugation, filtration, dialysis etc. This leads to a great amount of protein loss leading to lower yield of biologically active recombinant protein.

Traditionally, recombinant proteins are refolded to obtain their biologically active form by a process that starts with lysing of cells for isolating inclusion bodies through centrifugation of lysed cell solution. Thereafter, the isolated inclusion bodies are reconstituted in a buffer having a number of additives, denaturing agents, reducing agents, etc. Following the treatment of IBs with the buffer, the buffer and its components are removed through process of diafiltration, after which, the IBs are subjected to a unit process of refolding through one of the number of methods that are currently available e.g., oxidation method, sulphonation based methods, etc. The refolded protein is obtained in diluted form, which is concentrated by ultrafiltration and other relevant filtration techniques. The process of refolding is often governed by pH of the buffers, concentration of the additives, reducing agents, redox agents, denaturing agents used etc.

The process as described above is also represented, in terms of reaction kinetics, as two-state model of refolding of recombinant protein, which may be better represented by the diagram below:

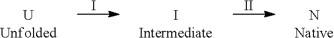

According to the current body of prior or existing arts, the kinetics of the folding process of a recombinant protein includes two stages. First stage includes formation of an intermediate I from the unfolded state U, the second stage that follows include formation of native state of the recombinant protein from the intermediate specie I. All of the current kinetic models that are derived on the basis of the processes that are available for refolding of protein depict that formation of native correctly folded recombinant protein is via formation of intermediates I. These structures being highly unstable have proclivity to get trapped into futile conformations to increase their stability, which results into lower overall yield of the correctly folded recombinant protein or the native protein N.

FIG. 1 illustrates kinetic and thermodynamic hypothesis, as developed based, on current processes available for protein refolding. As shown, the free energy profile of a protein as it starts to fold shows tendency to form stable native conformations, which may be called as Global Minimum and which depicts correct refolding of recombinant protein. However, due to formation of intermediates, as shown in the figure above, in order to attain stable conformations faster, the proteins usually get trapped in meta-stable state which may be termed as Local Minimum, which depicts incorrect refolding of recombinant protein. Due to higher tendency of the recombinant proteins, to move towards the Local Minimum energy state than the Global Minimum energy state, because of formation of unstable intermediates, overall yield of correctly refolded protein is low. Moreover, the local minimum energy state is different for the different unfolded protein molecule at different stages and environmental conditions leading to non-uniformity among the intermediates that are formed.

Many attempts have been made to optimise refolding of recombinant proteins in terms of optimising pH, optimising buffer content, but only a fewer exist that reduce the number of steps, required for refolding recombinant proteins, to a minimum possible. But none of the processes currently available, in the body of existing art, aim to improve the process of refolding of recombinant proteins through manipulating thermodynamic-kinetics of the process of refolding of the recombinant proteins.

Accordingly, there remains a need to develop a process of refolding the recombinant proteins that target the basic thermodynamics and reaction kinetics of protein refolding process and improve it or manipulate it to obtain higher yield of correctly refolded recombinant proteins.

### SUMMARY OF THE INVENTION

In view of the foregoing, the embodiments herein, provide a process for refolding of recombinant proteins through formation of stable intermediates.

In an aspect, a process for obtaining a refolded recombinant protein from an unfolded recombinant protein present in inclusion bodies isolated from wet cells harvested from a cell culture, is provided. The process includes a) reducing the inclusion bodies by treating the inclusion bodies with a reducing buffer, to obtain reduced inclusion bodies; b) obtaining stable first intermediate state I₁ of the unfolded recombinant protein present in the reduced inclusion bodies; and c) refolding the stable first intermediate state I₁ of the unfolded recombinant protein, present in the reduced inclusion bodies, in refolding buffer to obtain a second intermediate state I₂ that folds to produce the refolded recombinant protein. The stable first intermediate state I₁ of the unfolded recombinant protein present in the reduced inclusion bodies is obtained by trapping or exposing the reduced inclusion bodies in low pH environment and exchanging the reduced inclusion bodies against a denaturing agent at acidic pH. In one embodiment, the reduced inclusion bodies are exchanged against a denaturing agent at acidic pH via diafiltration to obtain stable first intermediate state I₁ of the unfolded recombinant protein present in the reduced inclusion bodies. Further, the stable first intermediate state I₁ of the unfolded recombinant protein in the reduced inclusion bodies is refolded at basic pH ranging between 7.5 and 11.5, preferably 10.5.

In another aspect, a process for obtaining a refolded recombinant protein from an unfolded recombinant protein present in inclusion bodies isolated from wet cells harvested from a cell culture, is provided. The process includes a) reducing the inclusion bodies by treating the inclusion bodies with a reducing buffer, to obtain reduced inclusion bodies; b) obtaining stable first intermediate state I₁ of the unfolded recombinant protein present in the reduced inclusion bodies by trapping or exposing the reduced inclusion bodies at a pH of 3.0 and exchanging the reduced inclusion bodies against a denaturing agent at a pH of 3.0; and c) refolding the stable first intermediate state I₁ of the unfolded recombinant protein, present in the reduced inclusion bodies, in refolding buffer to obtain a second intermediate state I₂ that folds to produce the refolded recombinant protein. The inclusion bodies are reduced at basic pH ranging between 7.5 and 11.5, preferably 10.5 and the stable first intermediate state I₁ of the unfolded recombinant protein in the reduced inclusion bodies is refolded at basic pH ranging between 7.5 and 11.5, preferably 10.5.

### BRIEF DESCRIPTION OF THE DRAWINGS

For a more complete understanding of the embodiments herein, reference should now be made to the embodiments illustrated in greater detail in the accompanying drawings and described below by way of examples:

FIG. 1 illustrates kinetic and thermodynamic hypothesis, as developed, based on current processes, in the body of existing art, available for protein refolding;

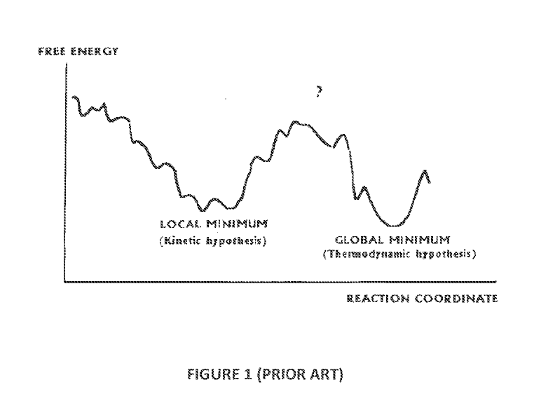

FIG. 2 illustrates a three stage model of refolding process of recombinant protein, according to an embodiment herein;

FIG. 3 illustrates a flowchart of the process for obtaining refolded recombinant protein, according to an embodiment herein; and

FIG. 4 illustrates comparison of RP-HPLC analysis for folding of a recombinant protein obtained using several methods known in the current body of prior arts with the method described in FIG. 3, according to an embodiment herein.

### DETAILED DESCRIPTION OF THE INVENTION

As required, detailed embodiments are disclosed herein; however, it is to be understood that the disclosed embodiments are merely exemplary, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language).

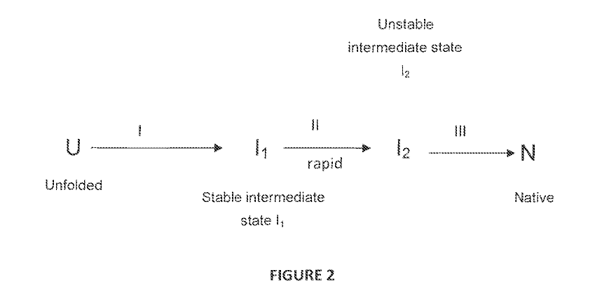

As mentioned, there is a need to develop a process for refolding the recombinant proteins that target the basic thermodynamics and reaction kinetics of protein refolding process and improve it or manipulate it to obtain higher yield of correctly refolded recombinant proteins. The embodiments, herein, describe a process that improves thermodynamic and reaction kinetics of the refolding of recombinant protein in a way that leads to higher yield of correctly refolded recombinant protein. The process provides a method of generating highly stable first intermediate states I₁ of a folding recombinant protein that leads to formation of a second intermediate state I₂. The thermodynamics and reaction kinetics of the refolding of the recombinant protein according to the process described herein are illustrated in FIG. 2. In one embodiment, the transition from the state I₁ to the state I₂ is rapid.

The process herein achieves a highly stable intermediate state such as I₁ before the unfolded protein is subjected to renaturing or refolding conditions, which leads to higher yield of correctly folded recombinant protein. Further, the process lowers the formation of unstable microstructures, which again leads to higher yield of correctly folded recombinant protein. Due to formation of a stable intermediate state such as I₁, the unfolded proteins have more tendency to go towards a Global Minimum energy or thermodynamic state. This also enables the unfolded proteins, undergoing conformational changes, to achieve a global minimum energy state, such as I₁, thereby, allowing a uniformity across unfolded protein molecules before they are subjected to refolding or renaturation conditions.

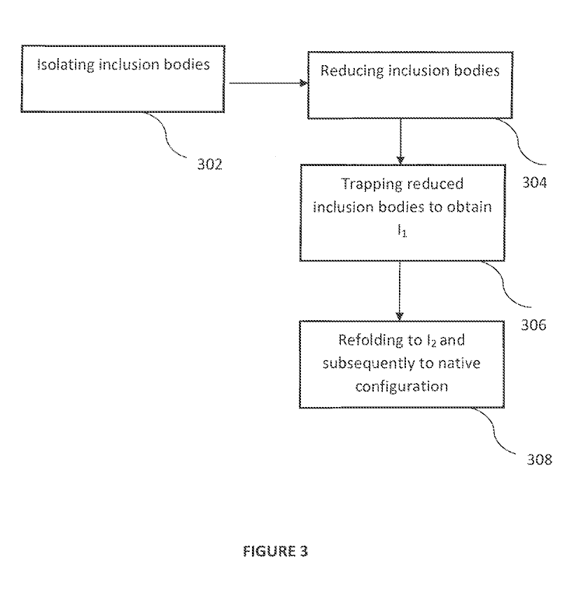

FIG. 3 illustrates a flowchart of the process for obtaining refolded recombinant protein, according to an embodiment herein. The process includes isolating inclusion bodies by lysing or homogenising cells, in step 302 and subjecting them to a reducing environment, having chaotropic and reducing agents, at basic pH in step 304 to obtain reduced inclusion bodies. This is followed by lowering the pH of reduced inclusion bodies, in step 306, to acidic pH with exchange against a strong denaturing agent in low pH conditions resulting in formation of a highly stable first intermediate state of reduced recombinant protein I₁ within the inclusion bodies. The low pH conditions assist in trapping the unfolded recombinant protein in first intermediate state I₁ which leads to stability of the state. In this step, the unfolded recombinant protein molecules, uniformly, achieve a global minimum energy state I₁. In step 308, the unfolded recombinant protein in the intermediate state I₁ is subjected to refolding conditions in a buffer that includes redox agents as well as low concentration of denaturing agents at a high pH environment. The treatment with high pH refolding conditions in step 308 leads to rapid formation of the second intermediate state I₂, where correct disulphide bonds are formed. This rapidly gives yield to correctly folded native form of the recombinant protein.

In one embodiment, the inclusion bodies are isolated in the presence of reducing agent i.e. wet cell slurry, meant for cell lysis, is first incubated with a reducing agent or reducing buffer and thereafter, lysed to obtain the inclusion bodies. The reducing buffer includes a denaturing agent or a chaotropic agent, a reducing agent and a buffering agent at basic pH. In one embodiment, the reducing buffer includes 0.25 mM dithiothrietol (DTT) and 1M urea.

The inclusion bodies may be isolated by a number of processes currently known in the art. In a preferred embodiment, the inclusion bodies are isolated by a continuous flow centrifugation. The inclusion bodies may also be isolated by the processes, selected from the group consisting of but not limited to, batch centrifugation, ion exchange chromatography, solvent extraction, affinity chromatography etc. Chaotropic agent may be selected from group consisting of, but not limiting to, urea, guanidine, arginine, sodium thiocyanate, SDS, sarkosyl, chlorides, nitrates, thiocyanates, cetylmethylammonium salts, trichloroacetates, chemical solvents such as DMSO, DMF) or strong anion exchange resins such as Q-Sepharose. Reducing agent may be selected from a group consisting of, but not limiting to, DTT, β-mercaptoethanol, cysteine, dithioerythritol (DTE), cysteamine, thioglycolate, glutathione, or sodium borohydride.

The process of conversion of unfolded proteins at the stable first intermediate state I₁ to the second intermediate state I₂ may be done by processes selected from group consisting of but not limiting to, oxidation folding, sulphonation, infinite dilution etc. In a preferred embodiment, the protein is refolded to state I₂ and finally to its native state by infinitely diluting the reduced inclusion bodies having unfolded recombinant protein in first intermediate state I₁ in a refolding buffer along with oxidation through spargers. No additional urea is needed to be added in the infinite dilution method of refolding since the residual urea in the reduced inclusion bodies having unfolded recombinant protein in first intermediate state I₁ is carried forward in the refolding buffer in lower concentration, as is required.

The refolding buffer may include a buffering agent such as sodium bicarbonate and a chelating agent such as EDTA at a basic pH. In one embodiment, the refolding buffer does not include any chelating agent. In yet another embodiment, the refolding buffer is deionised water maintained at a pH in range of 7.5 to 11.5. In a preferred embodiment, refolding buffer is any buffer or solution that is used in the unit process of refolding to obtain correctly folded recombinant protein. In one embodiment, the pH of refolding buffer is in range of 7.5 to 11.5, and more preferably at 10.5.

The volume of refolding buffer used for dilution is calculated to obtain a concentration of the unfolded recombinant protein in inclusion bodies in range of 0.1 g/L to 1 g/L. In one embodiment, the final concentration of the recombinant protein in the inclusion bodies in the refolding buffer is adjusted to 0.4 g/L. The final concentration of urea in the refolding buffer is adjusted such that it is not more than 3 M, but preferably less than 0.3 M. Further, the process of refolding is carried out in presence of atmospheric air which is introduced in the refolding mixture in form of bubbles through a number of spargers. The pressure of air is range of 0.01 bar to 2.5 bar. The temperature is maintained in the range of 4° C. to 25° C.

In one embodiment, a very low amount of reduced inclusion bodies having unfolded recombinant protein in first intermediate state I₁, are introduced into a stream of continuous refolding buffer flow in such a way that when introduced into the stream the resultant concentration of the target protein or the unfolded recombinant protein in the inclusion bodies is in range of 0.1 g/L-1 g/L and that of residual urea is not more than 3 M, preferably less than 0.3 M. In one embodiment, the reduced IBs are introduced into the refolding buffer in continuous flow arrangement such that concentration of the denaturing agent such as Urea in the reduced IBs is reduced below a concentration that is required for denaturing the IBs and, thereafter, the unfolded recombinant protein recombinant protein in first intermediate state I₁ in the reduced IBs is refolded into a biologically active refolded recombinant protein via formation of second intermediate I₂. In one embodiment, the refolding of the stable first intermediate state I₁ of the unfolded recombinant protein is done by any of plurality of methods selected from a group consisting of but not limiting to, infinite dilution, sulphonation, oxidation, air oxidation, redox based folding or on column folding.

The examples given below in a non-limiting manner will make it possible to better understand the embodiments herein.

### EXAMPLES

### Example 1

### Homogenisation of Wet Cells

After fermentation, the wet cells were harvested using a continuous centrifuge. The wet cells were diluted with 20× volumes of 20 mM Tris. The 20× volume of the Tris buffer was calculated on the basis of theoretical pellet weight of the wet cell mass. The slurry of the wet cells, obtained after dilution, was subjected to homogenisation by lysing cells using a cell disruptor up to 3 passes. The lysate obtained after the cell lysis contained inclusion bodies and lysed cells.

### Example 2

### Inclusion Body Isolation

The lysate was diluted with 20× volumes of phosphate buffer saline at a pH range of 7.3 to 7.5. The diluted lysate was subjected to centrifugation at 14000 G-15000 G force using a continuous centrifuge at a feed flow rate of 20-22 litres per hour. The resulting supernatant (S1) and pellet (P1) were collected separately. P1 was washed with phosphate buffered saline by centrifugation at 14000 G-15000 G force using the Westfalia continuous centrifuge. The resulting supernatant (S2) and the pellet (P2) was used for further inclusion body solubilisation.

### Example 3

### Inclusion Body Reduction

The inclusion bodies in form of pellet (P2) were reduced in a reducing buffer having 0.25 mM DTT, 8 M urea, and 2.5 mM glycine at pH ranging between 7.5 and 11.0. The volume of the reducing buffer was calculated such that the recombinant protein in the inclusion bodies had the final concentration of 15-18 g/L.

### Example 4

### Obtaining Intermediate State I₁ of the Unfolded Recombinant Protein in Inclusion Bodies

The reduced inclusion bodies were exposed to a buffer having acidic pH of 3.0 for a brief duration to obtain the intermediate state I₁ of the unfolded recombinant protein and thereafter, immediately processed further for refolding in a refolding buffer having 10 mM sodium bicarbonate, 1 mM EDTA at pH of 10.5. The volume of the buffer was calculated in a way to achieve a final concentration of the recombinant protein in range of 0.1 g/L-1 g/L of the inclusion bodies, whereas the concentration of urea is maintained at not more than 3.0 M, preferably lesser than 0.3 M. The pH of the refolding buffer was maintained at 10.5, with the refolding process carried out at 20° C.-25° C. in presence of oxygen at atmospheric pressure. The atmospheric air was introduced to the refolding mixture in form of bubbles through spargers.

### Example 5

### Effect of Formation of Stable Intermediate State I₁ at Acidic pH on Amount of Refolded Protein Obtained

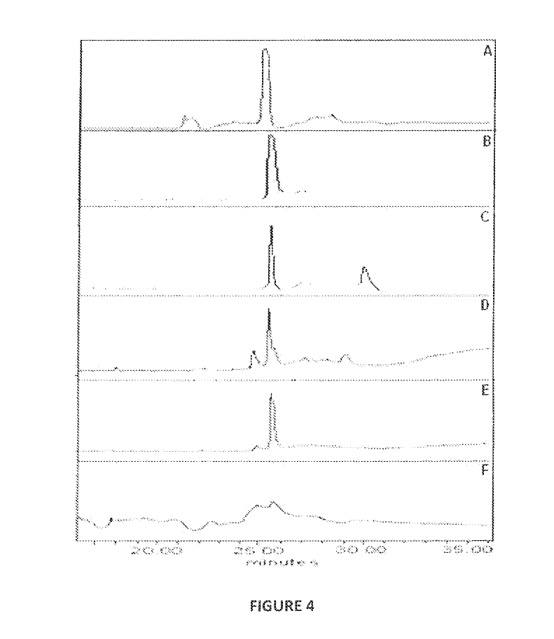

FIG. 4 illustrates comparison of RP-HPLC analysis for folding of a recombinant protein obtained using several methods known in the current body of prior art with the method described herein. 150 μg of folded recombinant protein, obtained through the methods marked A, B, C, D, E, and F was injected on a C 18 column. Method A corresponds to the process of refolding as described herein. Method B corresponds to the process described in Zhi-Song-Qiao et al (‘In Vitro Refolding of Human Proinsulin-Kinetic Intermediates, Putative Disulfide-forming pathway, Folding initiation site, and potential role of C-peptide in folding process’, J. Biol. Chem, 2003), in which the refolding occurs in the presence of redox shufflers; Method C corresponds to the process of refolding described by Yi Jin Hua and Zhang Yuan Xing (‘Refolding of the Fusion Protein of Recombinant Enterokinase Light Chain rEK_{L}’, Chinese Journal of Biotechnology, 2006) which is based on mixed disulphide based approach, and which does not allow generation of intermediaries; Method D corresponds to the process by Robert B. Mackin (‘Streamlined procedure for the production of normal and altered version of recombinant human proinsulin’, Protein Expression and Purification, 1998); and Method E corresponds to the processes described by Cowley et al (‘Expression, purification and characterization of recombinant human proinsulin’, FEBS Lett., 1997); and Method F corresponds to the method as described by Castallano-Serra et al (‘Expression and folding of an interleukin-2-proinsulin fusion protein and its conversion into insulin by a single step enzymatic removal of the C-peptide and the N-terminal fused sequence’, FEBS Lett, 1996). None of the methods among B, C, D E and F describes and teaches formation of intermediates as critical step towards achieving higher yield of production of refolded protein.

What we claim is:

1. A process for obtaining a refolded recombinant protein from an unfolded recombinant protein present in inclusion bodies isolated from wet cells harvested from a cell culture, comprising
a) reducing said inclusion bodies by treating said inclusion bodies with a reducing buffer, to obtain reduced inclusion bodies;
b) obtaining stable first intermediate state I₁ of said unfolded recombinant protein present in said reduced inclusion bodies; and
c) refolding said stable first intermediate state I₁ of said unfolded recombinant protein, present in said reduced inclusion bodies, in refolding buffer to obtain a second intermediate state I₂ that folds to produce said refolded recombinant protein.

2. The process of claim 1, wherein said inclusion bodies are isolated from said wet cells harvested from said cell culture by any of plurality of methods selected from a group comprising of but not limiting to, continuous centrifugation, batch centrifugation, ion exchange chromatography, affinity chromatography, and reverse phase chromatography.

3. The process of claim 1, wherein said stable first intermediate state I₁ of said unfolded recombinant protein present in said reduced inclusion bodies is obtained by exposing said reduced inclusion bodies in acidic pH environment.

4. The process of claim 3, further comprising exchanging sad reduced inclusion bodies against a denaturing agent at acidic pH.

5. The process of claim 1, wherein said inclusion bodies are reduced at basic pH ranging between 7.5 and 11.5, preferably 10.5.

6. The process of claim 1, wherein said refolding of said stable first intermediate state I₁ of said unfolded recombinant protein is done by any of plurality of methods selected from a group comprising of but not limiting to, infinite dilution, sulphonation, oxidation, air oxidation, redox based folding or on column folding.

7. The process of claim 1, wherein said stable first intermediate state I₁ of said unfolded recombinant protein in said reduced inclusion bodies is refolded at basic pH ranging between 7.5 and 11.5, preferably 10.5.

8. The process of claim 3, wherein said stable first intermediate state I₁ of said unfolded recombinant protein present in said reduced inclusion bodies is obtained by trapping or exposing said reduced inclusion bodies at a pH of 3.0.

9. The process of claim 8, further comprising exchanging said reduced inclusion bodies against a denaturing agent at a pH of 3.0.

10. A process for obtaining a refolded recombinant protein from an unfolded recombinant protein present in inclusion bodies isolated from wet cells harvested from a cell culture, comprising
a) reducing said inclusion bodies by treating said inclusion bodies with a reducing buffer, to obtain reduced inclusion bodies;
b) obtaining stable first intermediate state I₁ of said unfolded recombinant protein present in said reduced inclusion bodies by trapping or exposing said reduced inclusion bodies at a pH of 3.0; and
c) refolding said stable first intermediate state I₁ of said unfolded recombinant protein, present in said reduced inclusion bodies, in refolding buffer to obtain a second intermediate state I₂ that folds to produce said refolded recombinant protein.

11. The process of claim 10, wherein said inclusion bodies are reduced at basic pH ranging between 7.5 and 11.5, preferably 10.5 and said stable first intermediate state I₁ of said unfolded recombinant protein in said reduced inclusion bodies is refolded at basic pH ranging between 7.5 and 11.5, preferably 10.5.

12. The process of claim 10, further comprising exchanging said reduced inclusion bodies against a denaturing agent at a pH of 3.0 for obtaining stable first intermediate state I₁ of said unfolded recombinant protein present in said reduced inclusion bodies.
